(19) United States
(12) Patent Application Publication
(10) Pub. No.: US 2026/0256516 A1
(43) Pub. Date: Sep. 3, 2026

(54) EFFICIENT BIOLOGICAL TISSUE TREATMENT SYSTEMS AND METHODS

(71) Applicant: Sciton, Inc., Palo Alto, CA (US)

(72) Inventors: Alexander MAKOWSKI, Palo Alto, CA (US); Hartmuth HECHT, Palo Alto, CA (US); Daniel K. NEGUS, Palo Alto, CA (US)

(21) Appl. No.: 18/842,617

(22) PCT Filed: Mar. 7, 2023
(86) PCT No.: PCT/US2023/063843

§ 371 Date: Aug. 29, 2024

Related U.S. Application Data
(60) Provisional application No. 63/317,097, filed on Mar. 7, 2022.

(51) Int. Cl. A61B 18/20; A61B 18/00; A61B 90/00

(52) U.S. Cl. CPC A61B 18/20; A61B 90/37; A61B 2018/00642; A61B 2018/00702; A61B 2018/00791; A61B 2090/373

(57) ABSTRACT

Methods, systems, and computer storage media for the efficient treatment of biological tissue are disclosed. Such a method of treating tissue of a patient can comprise irradiating a region of interest (ROI) of the patient with one or more initial doses of electromagnetic radiation. The ROI comprises a matrix or array of sub regions of interest (sROIs) of biological tissue of the patient. The method further comprises determining a tissue response of one or more of the sROIs, generating a tissue response matrix from the tissue response of the one or more sROIs, and translating the tissue response matrix into a sub-clinical indicator matrix or treatment completion matrix for the one or more sROIs. The method also comprises irradiating one or more sROIs or no sROIs with one or more additional doses of electromagnetic radiation based at least in part on the sub-clinical indicator matrix.

### CROSS-REFERENCE TO RELATED APPLICATIONS

This application claims priority to U.S. Provisional Patent Application No. 63/317,097 filed on Mar. 7, 2022, the entire contents of which are incorporated herein by reference.

### FIELD

The technology described herein generally relates to the treatment of biological tissue (e.g., skin), and more specifically to efficient treatment of tissue that incorporates various imaging and control features.

### BACKGROUND

Some tissue (e.g., skin) conditions or diseases, for example Basal Cell Carcinoma (BCC), are rarely lethal but will continue growing if not treated. Amongst other tissue issues and diseases, BCC is the underlying cause of more than 5.4 million treatments per year in the US alone, making it a large treatment space as well as an economic burden.

Traditional treatment methods of tissue conditions such as BCC include intensive methods such as Mohs surgery, topicals, destruction, and excision. As an example, BCC treatment can require the death of cancer cells due to heat generation by a laser. BCC has the hallmark of being irregular in shape, hyperplastic, and having irregular, increased vascularity. The current state of technology of laser treatment of BCC requires clinicians to expose a patient to laser light based on guesswork, with post-treatment checking for a visual indication of treatment efficacy, such as greying of tissue that is associated with coagulative damage. This approach can be highly subjective and suffers under the learning curve of clinician training. Other existing approaches to treating BCC suffer from one or more other disadvantages. Current methods and systems for treatment of conditions other than BCC can also suffer from similar disadvantages such as lack of precision or lack of complete clinical efficacy.

Accordingly, there exists a therapeutic need for improved systems and methods of treatment that can provide both efficacy and reduction in risk of adverse events, such as overtreatment of target tissue and damage to healthy tissue. There is a need for improved treatment systems and methods that can achieve higher success rates, reduce clinician error, and reduce the need for additional treatments, all while being minimally invasive for the patient.

### SUMMARY

This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

Embodiments of the technology described herein are directed towards electromagnetic radiation-based (e.g., light-based) treatment of biological tissue, for instance that of a human patient in need thereof, that realizes improved precision and clinical results, reduces clinician error, and is less invasive for a patient, for example through the use of various imaging and control features. The disclosed technology can be used to treat various types of biological tissue of a patient (such as a human or animal patient) in need thereof, including, for instance, skin tissue or components of skin or diseased tissue on or within skin. Other organ tissue (e.g., liver tissue) may also be treated using methods and systems described herein.

Although often used in treatment of tissue pathology, doses of electromagnetic radiation and subsequent tissue responses are often mistakenly assumed to be effectively uniform in time. Actually, many sources of electromagnetic radiation are pulsed. Tissue response to pulsed doses of radiation can differ relative to pulse duration and intensity, as is the case in laser radiation. In an extreme example, for small long doses, reports of biostimulation have been made; moderate doses lead to well documented heating and/or coagulation; strong quick doses can cause tissue ablation; and extreme doses can even generate plasma. Even in routine examples, theoretical equations can be employed to design a static treatment paradigm that will neither over treat or under treat the tissue. This paradigm is tested and validated and distributed or identified as a “therapeutic window” for dosing, similar to doses of chemical medicine.

However, tissue response occurs on incredibly short time scales, and small fluctuations in tissue response are likely sub-clinical (that is, not easily observed and recorded by clinicians without instrumentation). Moreover, the inherent abnormal nature of pathological tissue results frequently in spatial heterogeneity within and between patients. The combined spatial heterogeneity of the pathology and time heterogeneity of the tissue response to electromagnetic radiation are likely responsible for some proportion of treatment failure when using therapeutic windows and static treatments alone. The present application describes, in some implementations, the integration of improved measurement systems into adaptive treatment methods that capture patient-specific tissue response and thus help ensure complete treatment.

In some embodiments, a method described herein may comprise monitoring biological tissue before, during and/or after treatment with one or more spatially resolved sensors. In some embodiments, multiple registered image sensors recognize and track physician-indicated region(s) of interest (herein “ROI”) designated for treatment. In some such embodiments, proper treatment can include spatially-resolved sensing and tracking of a matrix or array of sub-regions of interest (“sROI” as the singular or “sROIs” as the plural), where the sROIs are components of the physician-indicated treatment area (ROI). Specialized algorithms for signal and image processing can operate on the sensor data to determine, confirm and track the ROI, as well as simultaneously guiding the course of treatment in time, space and intensity based on patient-and tissue-specific responses to the treatment stimuli. Moreover, as described further herein, a “treatment completion integrator” can be provided, manifested in some embodiments as the joint tracking of a therapeutic window as well as the translation of tissue response measurements into logical fractional markers of treatment completion (e.g., posterior probability of cell death) using rate equations.

More generally, methods, systems, and computer storage media for the efficient treatment of biological tissue (e.g., skin or a component thereof) are described herein. In some embodiments, a method of treating a patient's biological tissue (e.g., skin) or a component thereof comprises irradiating a region of interest (ROI) of the patient with one or more initial doses of electromagnetic radiation, wherein the ROI comprises a matrix or array of sub regions of interest (sROIs) of biological tissue of the patient. The method further comprises determining a tissue response of one or more of the sROIs, generating a tissue response matrix from the tissue response of the one or more sROIs, and translating the tissue response matrix into a sub-clinical indicator matrix or treatment completion matrix for the one or more sROIs. The sub-clinical indicator matrix corresponds to a degree of treatment completion for the one or more sROIs. In some embodiments, the method further comprises irradiating one or more sROIs (or no sROIs) with one or more additional doses of electromagnetic radiation based at least in part on the sub-clinical indicator matrix.

Moreover, in some embodiments, the sub-clinical indicator matrix comprises an aggregation of degrees of treatment completion for the one or more sROIs over time. The sub-clinical indicator matrix may also be generated based on one or more rate functions, such as at least one of an Arrhenius function, a bioheat function, and a light transport function.

Additionally, in some cases, two or more of the irradiating, determining, generating, and translating steps occur simultaneously, continuously, and/or in real time. Further, in some implementations, the one or more additional doses of electromagnetic radiation have the same or different properties as the one or more initial doses.

Moreover, in some embodiments of a method described herein, the tissue response corresponds to a sensed temperature. Further, in some cases, the degree of treatment completion comprises achieving a benchmark temperature over time for one or more sROIs. In other instances, the degree of treatment completion comprises achieving cell death of the biological tissue of one or more sROIs. A tissue response described herein may also comprise or correspond to a spectral change, particularly a change in electromagnetic spectrum. For example, in some cases, a spectral change comprises a detected color (e.g., an electromagnetic radiation absorption and/or emission profile corresponding to a color perceived by an ordinary or healthy human eye) or a detected change in color. Other spectral changes may also be used, provided the spectral change is detectable as a tissue response within a method and/or system described herein.

Additionally, in some cases, each sROI corresponds to a unit-sized real physical location of the biological tissue with a known size and location relative to one or more other sROIs of the ROI. In some implementations, the ROI corresponds to a defined treatment area, which may be a physician-defined treatment area, as described further herein. A method described herein can also comprise irradiating the matrix of sROIs a sufficient number of times to complete the treatment.

It is to be understood that a method described herein can be carried out using various sources of electromagnetic radiation. For example, in some cases, the electromagnetic radiation is a beam of electromagnetic radiation such as a laser beam. In some such embodiments, the laser beam has an average wavelength λ between 700 nm and 1500 nm or between 900 and 1300 nm. In some implementations, the laser beam comprises an Nd: YAG laser beam.

In some embodiments, a method described herein further comprises imaging the ROI with an imaging device, such as a camera or other imaging device described herein. Moreover, in some cases, the ROI is imaged in real time.

Systems for treating biological tissue (such as skin) are also described herein. In some such embodiments, a system for treating biological tissue of a patient comprises a source of electromagnetic radiation to irradiate a region of interest (ROI) of the patient with one or more initial doses of electromagnetic radiation, wherein the ROI comprises a matrix or array of sub regions of interest (sROIs) of biological tissue of the patient. The system also comprises an imaging device and a tissue response detector to determine a tissue response of one or more of the sROIs based on the irradiation of the ROI. The imaging device can be configured to image one or more of the sROIs, including in real time if desired. In some cases, the tissue response detector can be configured to detect a temperature of one or more sROIs. It is further to be understood that, in some embodiments, the tissue response detector can detect a temperature at one sROI (or pixel in a two-dimensional image of the ROI) or at more than one sROI (or pixel) as a function of time. That is, the tissue response detector can be configured to detect a series of temperatures over time, at one sROI and/or at a plurality of sROIs. Further, in some embodiments described herein, one or more measured or detected sROI temperatures (or temporal profiles of one or more sROI temperatures) can be used to compute, estimate, or extrapolate a bulk temperature (as opposed to a surface temperature) of the ROI or sROI.

Additionally, in some embodiments, a system described herein further comprises a control device (or a network of control devices) to generate a tissue response matrix based on information received from the tissue response detector, and to translate the tissue response matrix into a sub-clinical indicator matrix or treatment completion matrix for the one or more sROIs. The sub-clinical indicator matrix can correspond to a degree of treatment completion for the one or more sROIs. Additionally, the control device (or network of control devices) can further signal the source of electromagnetic radiation to irradiate one or more sROIs or no sROIs with one or more additional doses of electromagnetic radiation based at least in part on the sub-clinical indicator matrix. The one or more additional doses of electromagnetic radiation can have the same or different properties as the one or more initial doses. In some embodiments of systems described herein, the sub-clinical indicator matrix comprises an aggregation of degrees of treatment completion for the one or more sROIs over time. Additionally, in some implementations, the sub-clinical indicator matrix is generated based on one or more rate functions, such as at least one of an Arrhenius function, a bioheat function, and a light transport function (or a derivative a light transport function).

In still another aspect, improved computer devices and storage media are also described herein. For example, computer storage media storing computer-useable instructions are described herein. Such a computer storage medium can be used to carry out a method and/or use a system described herein. For instance, in some cases, a computer storage medium stores computer-useable instructions that, when used by one or more computing devices, cause the one or more computing devices to treat biological tissue of a patient, the operations comprising: irradiating a region of interest (ROI) of the patient with one or more initial doses of electromagnetic radiation, wherein the ROI comprises a matrix or array of sub regions of interest (sROIs) of biological tissue of the patient; determining a tissue response of one or more of the sROIs; generating a tissue response matrix from the tissue response of the one or more sROIs; translating the tissue response matrix into a sub-clinical indicator matrix or treatment completion matrix for the one or more sROIs, the sub-clinical indicator matrix corresponding to a degree of treatment completion for the one or more sROIs; and irradiating one or more sROIs or no sROIs with one or more additional doses of electromagnetic radiation based at least in part on the sub-clinical indicator matrix. In some instances, the operations further comprise mapping a plurality of sROIs of the tissue. Moreover, in some embodiments, two or more of the irradiating, determining, generating, and translating steps occur simultaneously, continuously, and/or in real time.

Additional objects, advantages, and novel features of the invention will be set forth in part in the description which follows, and in part will become apparent to those skilled in the art upon examination of the following, or can be learned by practice of the invention.

### BRIEF DESCRIPTION OF THE DRAWINGS

Aspects of the technology presented herein are described in detail below with reference to the accompanying drawing figures, which are not necessarily drawn to scale.

FIG. 1 illustrates a schematic of an example operating environment and system for tissue treatment, in accordance with some implementations of the technology described herein.

FIG. 2 is a flow diagram showing a method for efficient tissue treatment, in accordance with some aspects of the technology described herein.

FIG. 3 is a block diagram of an example computing environment and/or device architecture in which some implementations of the present technology may be employed.

FIG. 4 illustrates a perspective view of a treatment device according to one embodiment described herein.

FIG. 5 illustrates a perspective view of a treatment device according to one embodiment described herein.

FIG. 6A illustrates a two-dimensional thermal image of an ROI comprising multiple sROIs, when treated in a manner not according to the present disclosure.

FIG. 6B illustrates a two-dimensional thermal image of an ROI comprising multiple sROIs, when treated in a manner according to the present disclosure.

FIG. 7A illustrates a plot of temperature as a function of distance for the ROI of FIG. 6A.

FIG. 7B illustrates a plot of temperature as a function of distance for the ROI of FIG. 6B.

FIG. 8A illustrates a two-dimensional output (detected image) of an RGB camera (imaging device) in accordance with a step of one embodiment of a method described herein.

FIG. 8B illustrates a two-dimensional output of a thermal camera (tissue response detector) in accordance with a step of one embodiment of a method described herein.

FIG. 9 illustrates a two-dimensional thermal image of a calibration setup used in one embodiment of a method described herein.

FIG. 10A illustrates a three-dimensional calibration plot used in one embodiment of a method described herein.

FIG. 10B illustrates a plot of error and outlier analysis used in one embodiment of a method described herein.

FIG. 10C illustrates a calibration polynomial used in one embodiment of a method described herein.

FIG. 11 illustrates a plot of the output of a calibration process according to one embodiment of a method described herein.

FIG. 12A illustrates an output image of a thermal camera (tissue response detector) used in one embodiment of a method described herein.

FIG. 12B illustrates an output image of an RGB camera (imaging device) used in one embodiment of a method described herein.

FIG. 13A illustrates an output image of a thermal camera (tissue response detector) used in one embodiment of a method described herein.

FIG. 13B illustrates an output image of an RGB camera (imaging device) used in one embodiment of a method described herein.

FIG. 14A illustrates an output image of a thermal camera (tissue response detector) used in one embodiment of a method described herein.

FIG. 14B illustrates an output image of an RGB camera (imaging device) used in one embodiment of a method described herein.

FIG. 15 illustrates an overlay or combination of a thermal image and an RGB image in accordance with one embodiment of a method described herein.

FIG. 16 illustrates a real-time display of an overlay or combination of a thermal image and an RGB image in accordance with one embodiment of a method described herein.

FIG. 17 illustrates a two-dimensional image provided by an imaging device during one step of one embodiment of a method described herein.

FIG. 18 illustrates two-dimensional images provided by an imaging device used in one embodiment of a method described herein.

FIG. 19A illustrates a two-dimensional image provided during one step of one embodiment of a method described herein.

FIG. 19B illustrates a two-dimensional image provided during one step of one embodiment of a method described herein.

FIG. 20A illustrates a two-dimensional image provided by an imaging device during one step of one embodiment of a method described herein.

FIG. 20B illustrates a two-dimensional image provided by an imaging device during one step of one embodiment of a method described herein.

FIG. 20C illustrates a two-dimensional image provided by an imaging device during one step of one embodiment of a method described herein.

FIG. 21A illustrates a computer illustration of an ROI according to one embodiment of a method described herein.

FIG. 21B illustrates a computer illustration of an ROI according to one embodiment of a method described herein.

FIG. 21C illustrates a composite image used in one embodiment of a method described herein.

FIG. 21D schematically illustrates a step of one embodiment of a method described herein.

FIG. 22 illustrates composite images of steps of a method according to one embodiment described herein.

FIG. 23 illustrates composite images of steps of a method according to one embodiment described herein.

FIG. 24 schematically illustrates a step of one embodiment of a method described herein.

FIG. 25 illustrates composite images of steps of a method according to one embodiment described herein.

### DETAILED DESCRIPTION

The subject matter of aspects of the present disclosure is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” can be used herein to connote different elements of methods employed, the terms should not be interpreted as implying any particular order among or between various steps disclosed herein unless and except when the order of individual steps is explicitly described.

Accordingly, embodiments described herein can be understood more readily by reference to the following detailed description, examples, and figures. Elements, features, apparatus, and methods described herein, however, are not limited to the specific embodiments presented in the detailed description, examples, and figures. It should be recognized that the exemplary embodiments herein are merely illustrative of the principles of the invention. Numerous modifications and adaptations will be readily apparent to those of skill in the art without departing from the spirit and scope of the invention

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9. All ranges disclosed herein are also to be considered to include the end points of the range, unless expressly stated otherwise. For example, a range of “between 5 and 10” or “5 to 10” or “5-10” should generally be considered to include the end points 5 and 10.

Further, when the phrase “up to” is used in connection with an amount or quantity; it is to be understood that the amount is at least a detectable amount or quantity. For example, a material present in an amount “up to” a specified amount can be present from a detectable amount and up to and including the specified amount.

Additionally, in any disclosed embodiment, the terms “substantially,” “approximately,” and “about” may be substituted with “within [a percentage] of” what is specified, where the percentage includes 0.1, 1, 5, and 10 percent.

Likewise, unless the context clearly demands otherwise, the use of the singular article “a” or “an” refers to “at least one.”

According to aspects of systems and methods described herein, efficient tissue treatment can be provided through the use of various imaging techniques and control systems that provide improvements over other forms of treatment. According to embodiments described herein, various imaging and control techniques can be implemented to track locations and sublocations corresponding to tissue or components of tissue that require treatment and measure tissue responses to treatment as it occurs (e.g., as tissue or components thereof are treated with electromagnetic radiation) and can be integrated over time to ensure complete and accurate treatment is achieved. In some embodiments it will be appreciated that tissue responses may be converted to probabilities of treatment completion over time that iterate based on a driving rate or rate function.

It will be appreciated that treatment, e.g., treatment of tissue or components of tissue, may be necessitated by pathology, which by definition means the tissue is “not normal” and as such will not have regular features. Abnormal vasculature and altered cell responses to stimuli are hallmarks of cancer, and one, singular (i.e., maximum) temperature measurement for treating a pathological region can be grossly insufficient for preferred tracking and guidance. Accordingly, the methods and systems described herein utilize, amongst other components, thermal cameras, visual (RGB) cameras, laser scanners, and computing engines and components which may implement tracking indicator generating sequences which enable the tracking of physical locations over time as well as tissue response (e.g., temperature) over time which can then be used to generate aggregate probabilities of cell death or completion of treatment per location. Additionally, as described further herein, a method described herein can be partially or fully automated if desired, including for implementation by a computer.

In some embodiments, one or more sROIs or target pixels of the tissue or a component of tissue corresponding to biological tissue of a patient can be irradiated with a dose of electromagnetic radiation (e.g., a first dose of a beam of electromagnetic radiation), via a beam of light, for instance a laser beam of light and/or broad band light. One or more target sROIs can be defined by a system described herein and/or by a clinician such that the sROIs are within a defined treatment area or ROI. In some embodiments, the treatment area is mapped by establishing sROIs within the defined treatment area or ROI.

It is to be understood that a “laser” can refer to a single lasing device that produces a single beam of laser light from a single lasing medium. The laser described herein can be a pulsed laser or a continuous wave (CW) laser. Moreover, when a pulsed laser is used, the laser can produce time-modulated pulses of the laser beam. For instance, in some cases, the laser beam comprises an ablative laser beam and the laser produces time-modulated pulses of the ablative laser beam. In other cases, the laser beam comprises a coagulative laser beam and the laser produces time-modulated pulses of the coagulative laser beam.

A laser or laser beam described herein can have any power and any peak or average emission wavelength not inconsistent with the objectives of this disclosure. For example, in some embodiments, a laser or laser beam of a device described herein has a peak or average emission wavelength in the infrared (IR) region of the electromagnetic spectrum. In some such cases, the laser or laser beam has a peak or average emission wavelength in the range of 1-4 μm, 1-3 μm, 2-4 μm, 2-3 μm, 8-12 μm, or 9-11 μm. For example, in some embodiments, the laser or laser beam comprises an erbium-doped yttrium aluminum garnet (Er: YAG) laser or laser beam or a neodymium-doped YAG (Nd: YAG) laser or laser beam having a peak or average emission wavelength of 2940 nm or 1064 nm. In other cases, the laser or laser beam comprises a carbon dioxide laser or laser beam. A laser beam described herein can also have a peak or average emission wavelength in the visible region of the electromagnetic spectrum. Non-limiting examples of peak or average emission wavelengths suitable for use in some embodiments described herein include 532 nm, 695 nm, 755 nm, 1064 nm, and 1470 nm (e.g., for non-ablative application), or 2940 nm (e.g., for ablative application). Further, in some instances, a laser or laser beam of a device described herein has an average power of 1 to 10 W (e.g., when used for non-ablation) or 50 to 200 W (e.g., when used for ablation).

Moreover, the spot size of a laser beam produced by a laser described herein may also vary. Any spot size not inconsistent with the objectives of the disclosure may be used. In some cases, for instance, the spot size is 0.1-10 mm, 0.1-1 mm, 0.1-0.5 mm, 0.5-5 mm, 1-10 mm, or 1-5 mm. Other spot sizes may also be used. The laser beam can have any cross-sectional shape not inconsistent with the objectives of this disclosure.

The dose of electromagnetic radiation can have various properties, such as a determined intensity, fluence and/or duration. In response to the dose of electromagnetic radiation, a given sROI or plurality of sROIs that is irradiated and is associated with the tissue or component of tissue can produce a tissue response. For example, in some instances the tissue response may be heat generation, measured as thermal response or surface temperature. In other instances, the tissue response could be fluorescence or other luminescence, altered light absorption or scattering, or change in electrical and/or mechanical properties, among other properties. In some instances, the thermal response of a target sROI (or plurality of sROIs) can be directly measured or otherwise sensed, for instance via an infrared (IR) optical input or imaging device. In some cases, the tissue response may be indirectly measured (e.g., using a surface temperature analogue).

Based on the determined and/or measured tissue response corresponding to the target sROI (or plurality or matrix or array of sROIs), a sub-clinical indicator matrix can be generated. In some instances, the sub-clinical indicator matrix can correspond to a probability associated with a treatment procedure of the tissue or a component of tissue. In some instances, the generated sub-clinical indicator matrix can correspond to a degree of treatment completion for the relevant sROIs, for instance the sub-clinical indicator matrix can correspond to greater than 80% cell death of biological tissue associated with a biological tissue of one or more sROIs, or to greater than 90% cell death, or up to 100% cell death. In some instances, treatment completion comprises achieving a benchmark temperature or temperature profile within a “therapeutic window” one or more sROIs. Such a “therapeutic window” can be described or identified as the intensity of tissue response that is sufficient to begin a biological alteration to tissue function but not sufficiently intense as to cause an adverse event.

The sub-clinical indicator matrix can be generated using methods, systems, and components described herein. In some embodiments, the sub-clinical indicator matrix can be generated based on a rate function or otherwise using a rate function. In this way, a rate function can utilize the determined tissue response to then generate a sub-clinical indicator matrix. In some example embodiments, the rate function can include an Arrhenius function, a bioheat function, and a light transport function, amongst others. In some instances, the rate function comprises a derivative of a light transport function. Utilizing the sub-clinical indicator matrix, one or more additional doses of the electromagnetic radiation can be delivered to one or more sROIs or to no sROI. The use of Arrhenius functions is described, for example, in W. C. Dewey, “Arrhenius relationships from the molecule and cell to the clinic,” Int. J. Hyperthermia, February 2009; 25(1): 3-20. Thermal dosing of tissue is described, for example, in Dewhirst et al., “Thermal Does Requirement for Tissue Effect: Experimental and Clinical Findings,” Proc SPIE Int Soc Opt Eng. 2003 Jun. 2; 4954:37. As described herein, relationships of thermal dose to tissue effect can be used in some embodiments of the present technology to provide a sub-clinical indicator matrix or treatment completion matrix for an ROI or for one or more sROIs.

In some embodiments, a first dose can have a first fluence (f1) and a first duration (t1). An additional dose can have a second fluence (f2) and a second duration (t2). In some other instances, a dose can have a fluence (fn) and a duration (tn). It will be appreciated that a first fluence can be the same as a second, an additional, or another fluence, and a first duration can be the same as a second, an additional, or another duration. In such embodiments, one or more of the rate based functions or equations, the therapeutic window monitoring, and the tissue response measurements may factor in to the alteration of the intensity, fluence, or duration of the treatment doses during the treatment.

In some embodiments, as stated above, the source of electrogmatic radiation can be a laser beam, for instance having an average wavelength λ between 700 and 1500 nm. In some other instances, the laser beam can have an average wavelength λ between 900 and 1300 nm. In some embodiments, the laser is a Nd: YAG laser.

Other electromagnetic radiation sources may be used in conjunction with the present technology. As understood by one of ordinary skill in the art, the terms “BBL” source and “BBL beam” can refer to a source and beam, respectively, of intense, broad-spectrum pulses of light, including as defined and approved by the U.S. Food and Drug Administration. More particularly, a BBL beam produced by a BBL source can comprise pulses of non-coherent or non-laser light having a wavelength from 500 nm to 1200 nm, as described, for instance, in Raulin et al., “IPL technology: a review,” Lasers Surg. Med. 2003, 32: 78-87.

Any laser, BBL source, laser beam, or BBL beam not inconsistent with the objectives of this disclosure can be used. Moreover, the choice of laser, BBL source, or laser or BBL beam can be based on a desired effect of the laser or BBL beam and/or on a desired target of the laser or BBL beam. A BBL source described herein generally produces a pulsed light output. In some cases, the BBL source comprises a xenon gas-filled chamber. In such instances, the BBL source can produce a BBL beam by the application of bursts or pulses of electrical current through the xenon-containing chamber.

Imaging as it relates to the present technology can be carried out with any imaging system not inconsistent with the objectives of the present disclosure. For example, in some cases, the imaging system can comprise an optical imaging system, such as a spectrophotometer, a thermal camera, ultrasound, an optical coherence tomography (OCT) system, a multi-photon imaging system, a reflectance confocal microscopy (RCM) system or any other imaging system not inconsistent with the objectives of this disclosure. In some instances, a selectively reflective optical element can be configured to reflect both an outgoing beam and a return signal of the optical imaging system to permit the imaging system to both “probe” a target area and also receive a return signal from the target area. For instance, in the case of an OCT imaging system, the imaging system can comprise an OCT pilot or probing beam generator and an OCT detector.

Methods described herein, in some embodiments, can also comprise using imaging to identify a treatment area (e.g., a pre-defined treatment area) to which the method is applied. For example, in some cases, a treatment area may be labeled, marked, or delineated by a service provider or clinician (e.g., a physician or other medical care provider). In some such embodiments, a method described herein comprises labeling, marking, delineating, or otherwise identifying a treatment area of a patient, prior to carrying out initial therapeutic irradiation of an ROI or sROI. Such labeling, marking, delineating, or other identification can be carried out in any manner not inconsistent with the technical objectives of the present disclosure. For instance, in some embodiments, a perimeter is drawn around a desired treatment area using a marker or pen (e.g., for identifying a treatment area using ink disposed on skin) or using digital equipment (e.g., a stylus for identifying a treatment area digitally, such as by forming a perimeter on an image of the treatment area or patient, such as may be provided by a real-time camera image).

It is also possible, in some cases, to label a desired treatment area in other ways. For example, in some cases, labeling comprises applying a contrast agent or dye to the patient or a portion of the patient. In some instances, the contrast agent or dye migrates to a desired structure of the patient (e.g., a component of skin or a lesion) due to biological action of the patient or other action induced by application of the contrast agent or dye. In some embodiments, the contrast agent is charged or ionic. A contrast agent can also be an organic contrast agent or dye. One non-limiting example of a contrast agent that can be used in a method described herein includes methylene blue. Other contrast agents may also be used. In addition, in some cases, applying a contrast agent to the patient comprises applying a composition (such as a solution, cream, or paste) containing the contrast agent to the surface of the skin. Moreover, in some embodiments, a contrast agent is applied to the skin electrophoretically or using iontophoresis. For instance, in some cases, the contrast agent is delivered through the local application of an electrical current to the skin. The use of an electrical current or voltage may be especially preferred for labeling pores with a contrast agent such as methylene blue. It is also possible to use intravenous or other systemic injection of a contrast agent or dye. Specific components, constituents, or structures of a patient may be labeled in other manners as well, as understood by one of ordinary skill in the art.

After marking, delineating, or labeling of a desired treatment area is carried out, a method described herein, in some cases, can comprise detecting the desired treatment area prior to irradiating an ROI or sROI as described herein. In some cases, such detection is carried out using imaging software and/or hardware, possibly in combination with a controller or computer. Thus, in some embodiments, computer-aided or computer-implemented methods are described herein. In some cases, such a method is for identifying and determining characteristics of a desired treatment area or ROI so as to guide the appropriate delivery of irradiation to one or more sROIs as described herein. In some instances, such a method comprises capturing one or more images with at least one camera (such as a digital camera described herein). The camera can have a fixed or unvarying focal length. Additionally, if a plurality of cameras is used, each camera can have a fixed focal length, though the fixed focal lengths of the plurality of cameras can vary from one another. A method described herein can further comprise compensating for one or more optical distortions of the optical path of the camera that may be present. In some cases, the method also comprises cropping one or more of the one or more images, as needed or desired, and retaining only the portion of any cropped images that is relevant to therapy performed by the method. In some embodiments, the method further comprises identifying therapeutically pertinent sROIs. Such sROIs, in some instances, are identified by their spatial location, size, color, estimated depth beneath the surface of the skin, and/or estimated angle of shaft, in the event there is a shaft. A method described herein can further comprise mapping sROIs to a mechanical model of a positioning system of a light source, such that the position of the light source (and/or a beam of light or electromagnetic radiation provided by the light source) is known relative to the position of the relevant sROIs. Such a method can further comprise transmitting the sROI characteristics (e.g., location) to a controller of the light source so that a light beam can be directed to one or more sROIs as desired by a user. It is to be understood that one or more of the foregoing steps of a computer-implemented method can be carried out using hardware and/software described herein.

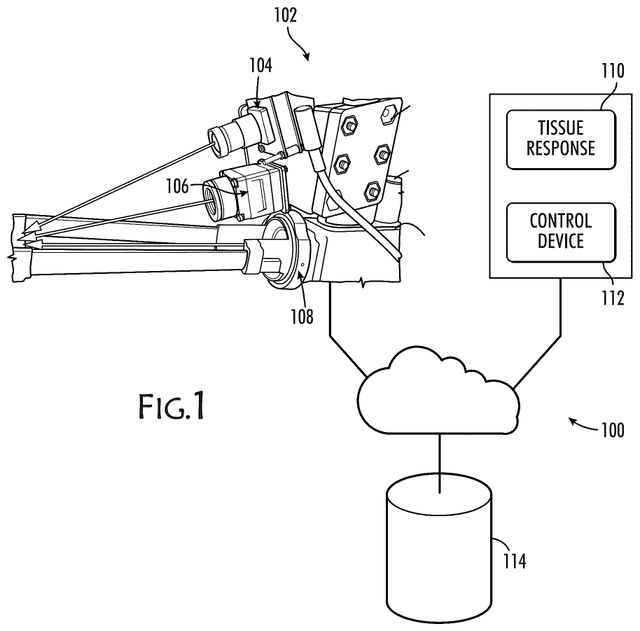

Referring now to the figures, with reference to FIG. 1, FIG. 1 depicts aspects of an efficient tissue treatment system 100 in accordance with various embodiments of the present technology. Tissue treatment system 100 can include a plurality of devices, components, engines, and/or modules. A treatment device 102 can include, amongst other components, one or more imaging devices, such as tissue response detector 104 and imaging device 106 and further a source of electromagnetic radiation 108. In some embodiments, tissue response detector 104 is an infrared (IR) or thermal camera and imaging device 106 is an RGB or other visible light camera. An imaging device can include, but is not limited to, any one or more of ultrasounds, x-rays, cameras, sensors, or systems thereof. Sensors, in some embodiments, are sensors of light. In other embodiments, an imaging device 106 includes devices capable of rendering 3-dimensional data and apply characteristics to the 3-dimensional data, such as hardness or toughness, temperature, or other characteristics. Arrows extending from tissue response detector 104, imaging device 106, and source of electromagnetic radiation 108 in FIG. 1 schematically illustrate the signals moving to/from the tissue response detector 104, imaging device 106, and source of electromagnetic radiation 108, ‘converging’ on an ROI (not expressly illustrated) adjacent to an applicator component or ‘standoff’ of the treatment device 102. Exemplary applicator components or ‘standoffs’are further described hereinbelow.

Tissue treatment system 100 can additionally include a tissue response detection engine 110 and a control device (or network of control devices) 112 which can be implemented to generate a sub-clinical indicator matrix. Any control device (or network of control devices) 112 not inconsistent with the technical objectives of the present disclosure may be used. For example, in some cases, a control device 112 comprises computing hardware and/or software. In some embodiments, control device 112 is a special purpose computer configured to improve the technological field of imaging and light therapy diagnoses and/or treatments. Such improvements are manifested in terms of improved targeting and light treatment of individual sROIs (e.g., individual skin cells, cancer cells, etc.). It is contemplated that a control device 112 can be a separate device (e.g., physically separate, non-integrated, discrete), entity, or component. However, a system comprising a control device 112 that is integrated with one or more other components described herein is also contemplated.

In addition, in some cases, a control device 112 can comprise one special purpose computer or a network of more than one special purpose computer. Moreover, in some instances, more than one control device 112 is used. For example, in some implementations, an imaging device described herein can be associated with a first control device (which may be denoted as a ‘slave’ device, for instance), and a source of electromagnetic radiation (e.g., a laser or BBL source) can be associated with a second control device (which may be denoted as a ‘master’ device, for instance), and the first and second control devices can coordinate with one another in accordance with steps of a method described herein. Likewise, a control device 112 described herein can provide signals either directly or indirectly to other components of a system described herein.

System 100 can further include a mapping component. As depicted, tissue treatment system 100 comprises a content repository 114, which can be a plurality of repositories, which can be in operable communication with treatment device 102 and any associated engines or modules.

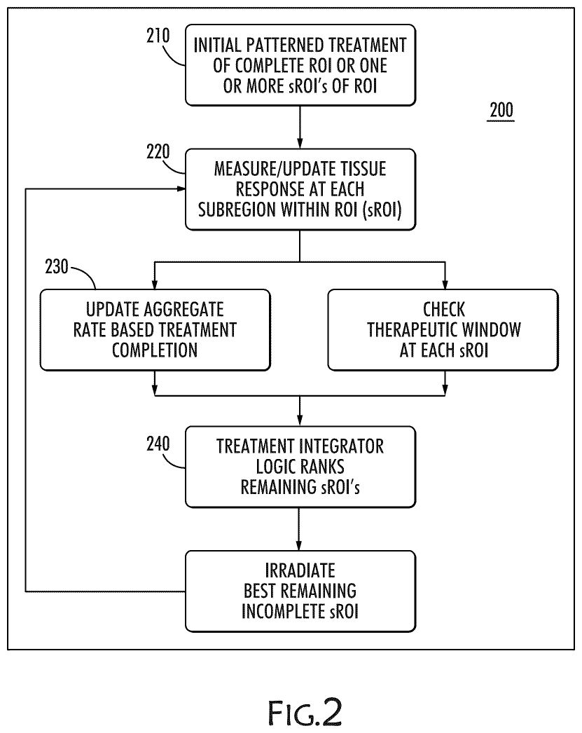

Turning now to FIG. 2, a flow diagram is provided illustrating one example method 200 for treating tissue or a component of tissue. It is contemplated that method 200 and other methods described herein are not limited to those illustrated and can incorporate other blocks or steps at any point in the method in accordance with the present disclosure. At step 210 one or more sROIs of an ROI (or the entire ROI) is irradiated with one or more initial doses of electromagnetic radiation, wherein the one or more sROIs comprises biological tissue of a patient. At step 220 a tissue response for the one or more sROIs is determined, for example a thermal response. The tissue response(s) can form a tissue response matrix for the sROIs and, based on the tissue response, at step 230 a sub-clinical indicator matrix corresponding to a degree of treatment completion for the one or more sROIs can be generated. Subsequently, at step 240 and based at least in part on the generated sub-clinical indicator matrix, a decision can be made regarding whether or not to further irradiate a specific sROI, a plurality of sROIs, or no sROIs.

Accordingly, various aspects of the technology for efficiently treating tissue are described. It is understood that various features, sub-combinations, and modifications of the embodiments described herein are of utility and can be employed in other embodiments without reference to other features or sub-combinations. Moreover, the order and sequences of steps shown in the example method 200 are not meant to limit the scope of the present invention in any way, and the steps can occur in a variety of different sequences within various embodiments. Such variations and combinations thereof are also contemplated to be within the scope of embodiments of the invention.

In some embodiments, for example, a method of treating tissue or a component of tissue comprises removing, ablating, vaporizing, destroying, or otherwise treating the tissue or component of tissue within a target area of a device described herein, including by directing a laser or BBL beam onto the tissue or component of tissue from the device. Moreover, as described further herein, a method described herein can comprise labeling, imaging, detecting, and/or mapping the tissue or component of tissue prior to (or substantially simultaneously with) directing a laser or BBL beam onto the tissue or component of tissue for treatment purposes. Alternatively, in other instances, the structure of tissue is not labeled but is instead imaged or detected in its “native” or natural state, without first labeling the structure. It may be especially desirable to omit a labeling step when the relevant structure of tissue is visually discernible, including in a facile manner, without the use of a labeling agent.

Methods described herein also comprise directing or applying electromagnetic radiation to tissue or a specific component, constituent, or structure of tissue. Electromagnetic radiation can be applied to tissue or a component, constituent, or structure of tissue in any manner not inconsistent with the objectives of the present disclosure. For example, in some cases, the electromagnetic radiation is a laser or BBL beam or radiofrequency beam produced by a device described herein. A high-frequency ultrasound beam may also be used instead of electromagnetic radiation. Any such laser, BBL source, or other source of electromagnetic radiation (or alternatively of high-frequency ultrasound) not inconsistent with the objectives of the present disclosure may be used. For example, in some cases, the electromagnetic radiation is applied by a laser or BBL source coupled to a camera (or other imaging system) and a controller.

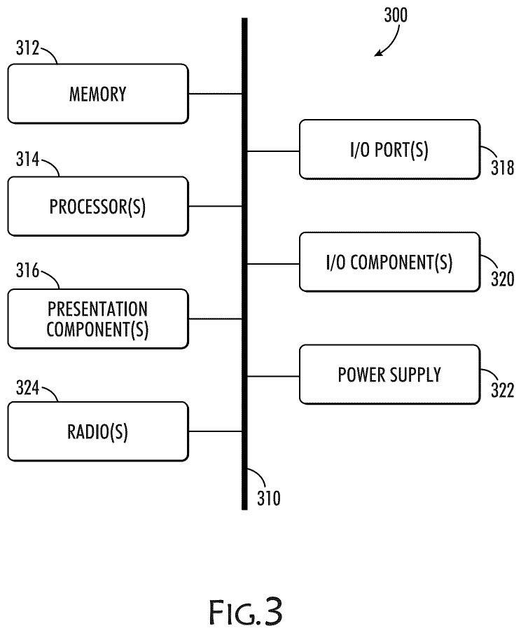

FIG. 3 provides an illustrative operating environment for implementing embodiments of the present invention is shown and designated generally as computing device 300. Computing device 300 is merely one example of a suitable computing environment and is not intended to suggest any limitation as to the scope of use or functionality of the invention. Neither should the computing device 300 be interpreted as having any dependency or requirement relating to any one or combination of components illustrated.

Embodiments of the invention can be described in the general context of computer code or machine-useable instructions, including computer-executable instructions such as program modules, being executed by a computer or other machine (virtual or otherwise), such as a smartphone or other handheld device. Generally, program modules, or engines, including routines, programs, objects, components, data structures etc., refer to code that perform particular tasks or implement particular abstract data types. Embodiments of the invention can be practiced in a variety of system configurations, including hand-held devices, consumer electronics, general-purpose computers, more specialized computing devices, etc. Embodiments of the invention can also be practiced in distributed computing environments where tasks are performed by remote-processing devices that are linked through a communications network.

With reference to FIG. 3, computing device 300 includes a bus 310 that directly or indirectly couples the following devices: memory 312, one or more processors 314, one or more presentation components 316, input/output ports 318, input/output components 320, and an illustrative power supply 322. In some embodiments, devices described herein utilize wired and rechargeable batteries and power supplies. Bus 310 represents what can be one or more busses (such as an address bus, data bus or combination thereof). Although the various blocks of FIG. 3 are shown with clearly delineated lines for the sake of clarity, in reality, such delineations are not so clear and these lines can overlap. For example, one can consider a presentation component such as a display device to be an I/O component as well. Also, processors generally have memory in the form of cache. It is recognized that such is the nature of the art, and reiterate that the diagram of FIG. 3 is merely illustrative of an example computing device that can be used in connection with one or more embodiments of the present disclosure. Distinction is not made between such categories as “workstation,” “server,” “laptop,” “hand-held device,” etc., as all are contemplated within the scope of FIG. 3 and reference to “computing device.”

Computing device 300 typically includes a variety of computer-readable media. Computer-readable media can be any available media that can be accessed by computing device 300, and includes both volatile and non-volatile media, removable and non-removable media. By way of example, and not limitation, computer-readable media can comprise computer storage media and communication media.

Computer storage media include volatile and non-volatile, removable and non-removable media implemented in any method or technology for storage of information such as computer-readable instructions, data structures, program modules, or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by computing device 300. Computer storage media excludes signals per se.

Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner at to encode information in the signal. By way of example, and not limitation, communication media includes wired media such as a wired network or direct-wired connection, and wireless media such as acoustic, RF, infrared, NFC, Bluetooth and other wireless media. Combinations of any of the above should also be included within the scope of computer-readable media.

Memory 312 includes computer storage media in the form of volatile and/or non-volatile memory. As depicted, memory 312 includes instructions 324, when executed by processor(s) 1014 are configured to cause the computing device to perform any of the operations described herein, in reference to the above discussed figures, or to implement any program modules described herein. The memory can be removable, non-removable, or a combination thereof. Illustrative hardware devices include solid-state memory, hard drives, optical-disc drives, etc. Computing device 300 includes one or more processors that read data from various entities such as memory 312 or I/O components 320. Presentation component(s) 316 present data indications to a user or other device. Illustrative presentation components include a display device, speaker, printing component, vibrating component, etc.

I/O ports 318 allow computing device 300 to be logically coupled to other devices including I/O components 320, some of which can be built in. Illustrative components include a microphone, joystick, directional pad, monitor, scanner, printer, wireless device, battery, etc.

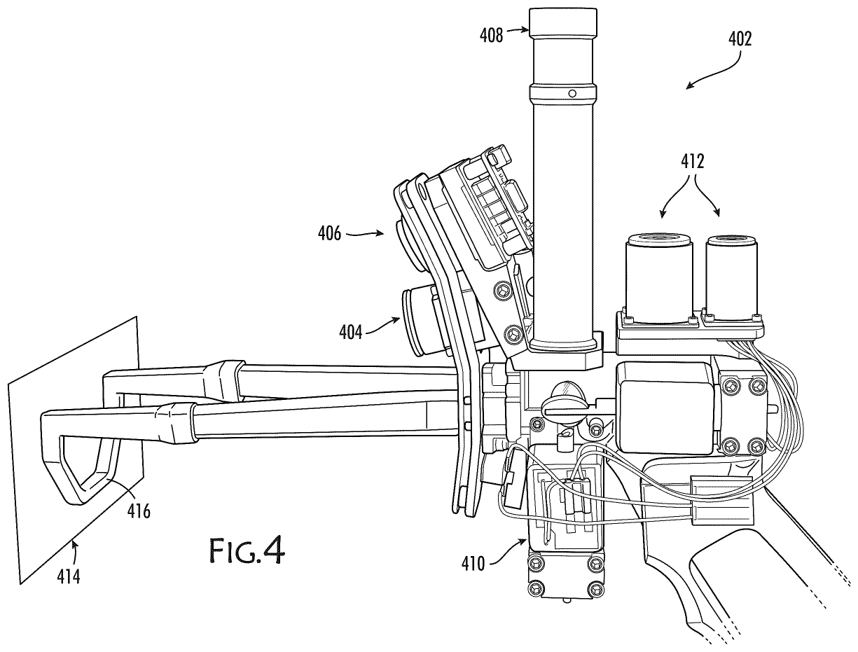

Turning now to FIG. 4, FIG. 4 illustrates a perspective view of a treatment device 402 according to one embodiment described herein. The treatment device 402 comprises a tissue response detector 404 and imaging device 406. A source of electromagnetic radiation 408 is also present in or coupled to the treatment device 402. In the embodiment of FIG. 4, the source of electromagnetic radiation 408 is a laser beam input. The treatment device 402 also comprises a steering module or controller 410 for steering or directing the laser beam provided by source 408. The device 402 further comprises connectors 412 for energy supply and communication (e.g., for coupling the device 402 to control systems and other system components described herein, not illustrated in FIG. 4). In addition, the treatment device 402 comprises a u-shaped applicator component 416 that can be used to align or position the treatment device 402 with reference to a plane 414. The plane 414 can be defined by the surface of an ROI or treatment area. For example, the plane 414 can be defined by the skin of a patient.

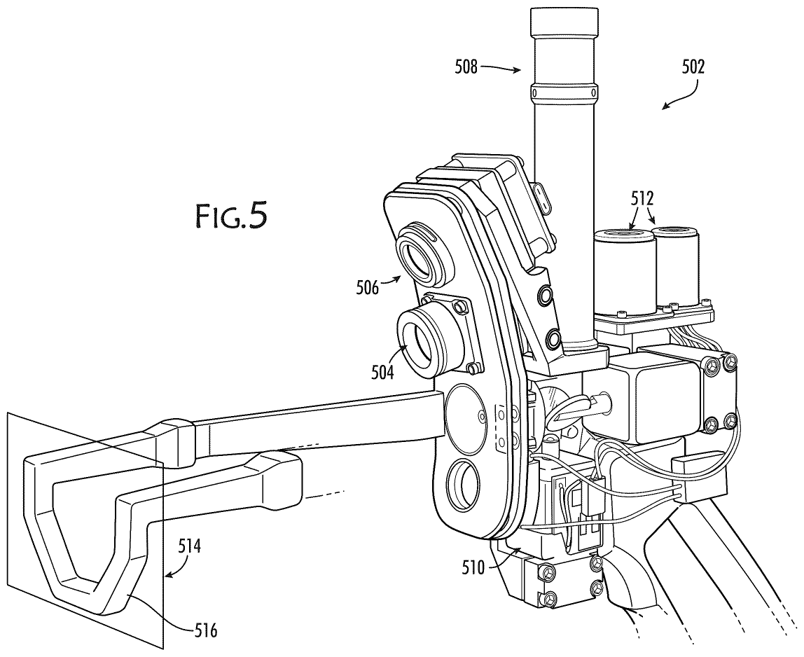

FIG. 5 illustrates a different perspective view of a treatment device 502 that is analogous or similar to the treatment device 402 of FIG. 4. The treatment device 502 of FIG. 5 comprises a tissue response detector 504 and imaging device 506. A source of electromagnetic radiation 508 is also present in the treatment device 502. In the embodiment of FIG. 5, the source of electromagnetic radiation 508 is a laser beam input. The treatment device 502 also comprises a steering module or controller 510 for steering or directing the laser beam provided by source 508. The device 502 further comprises connectors 512 for energy supply and communication (e.g., for coupling to control systems and other system components described herein, not illustrated in FIG. 5). In addition, the treatment device 502 comprises a u-shaped applicator component 516 that can be used to align or position the treatment device 502 with reference to a plane 514. The plane 514 can be defined by the surface of an ROI or treatment area. For example, the plane 514 can be defined by the skin of a patient. It should further be noted that the two round apertures 518 and 520 below detector 504 in FIG. 5 correspond to where the laser beam from source 508 exits toward the plane 514 (aperture 518 immediately below detector 504), and to a high power illumination source (which can be an LED source, for instance) that provides constant visible light for aiding the physician and the imaging device 506. This illumination source is the aperture 520 below the laser aperture 518 in FIG. 5.

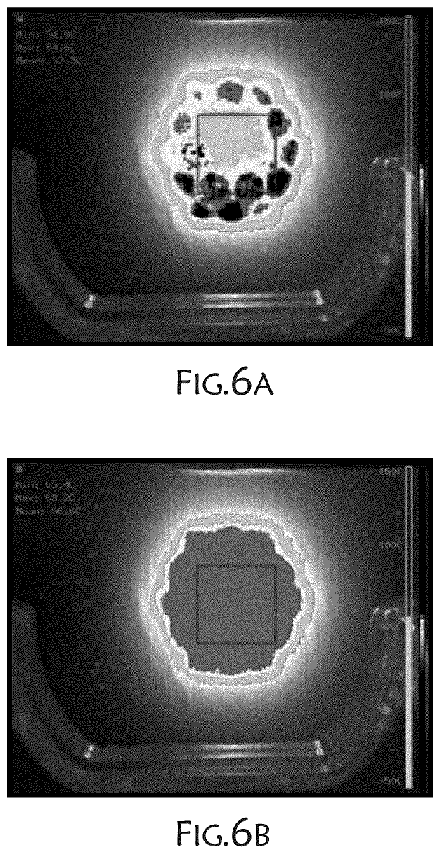

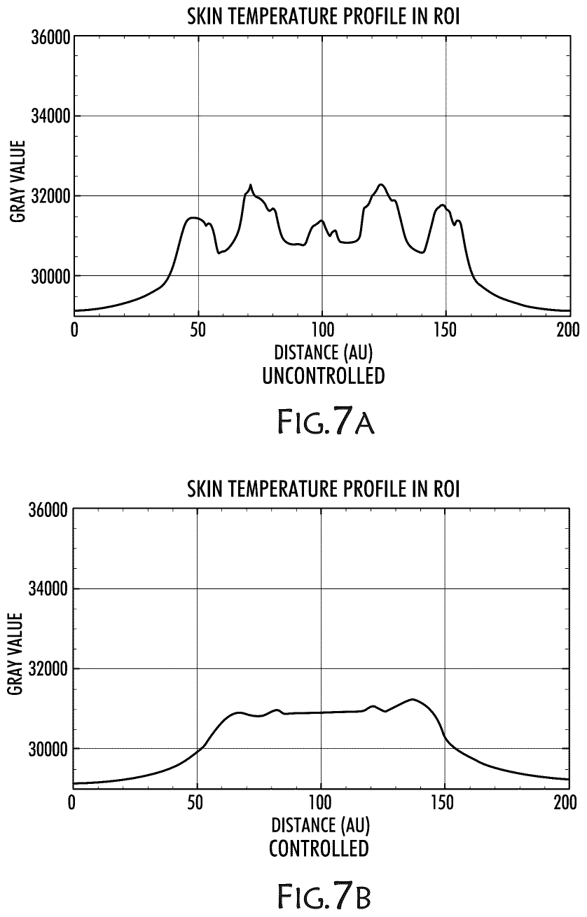

Systems, devices, and methods described herein (including, e.g., in FIGS. 1-5) can be used to provide improved treatment to a patient in need thereof. Non-limiting example advantages of systems, devices, and methods described herein are illustrated in FIG. 6 and FIG. 7.

With reference to FIG. 6 and FIG. 7, skin temperature profiles in a treatment plane (e.g., ROI) caused by laser impact, irradiation, or dosing are illustrated. Specifically, FIG. 6A and FIG. 7A each illustrate a skin temperature profile obtained by a previous method, which does not control treatment in the manner described in the present disclosure. FIG. 6A is a thermal image of a treated area or ROI, in which temperatures are indicated in grayscale. Darker gray represents a higher temperature at a given pixel or spot (or sROI), and lighter gray represents a lower temperature at a given pixel or spot (or sROI). As can be seen in FIG. 6A (the ‘uncontrolled’ case), treatment according to previous methods provides a wide range of temperatures from pixel to pixel or spot to spot (or sROI to sROI), even though the intended outcome of the method is uniform treatment across all sROIs. In contrast, methods according to the present disclosure (the ‘controlled’ case) provide even or uniform treatment that is substantially improved due to methods and systems described herein. As seen in FIG. 6B (which, like FIG. 6A, is a thermal image of a treated area or ROI in grayscale), temperature from pixel to pixel or spot to spot in the ROI (that is, from sROI to sROI) is much more uniform as compared to FIG. 6A. Moreover, the improved uniformity can be seen in the curves of FIG. 7A (uncontrolled case) and FIG. 7B (controlled case). FIGS. 7A and 7B are each plots of temperature as a function of distance across the ROI, that is, moving from one sROI to another sROI.

Embodiments described herein can be further understood by reference to the following additional Examples. Elements, apparatus, and methods described herein, however, are not limited to any specific embodiment presented in the Examples. It should be recognized that these are merely illustrative of some principles of this disclosure, and are non-limiting. Numerous modifications and adaptations will be readily apparent without departing from the spirit and scope of the disclosure.

### EXAMPLES

In one example embodiment, the treatment of BCC is provided, which comprises spatially resolved, image guided, closed-loop-control tissue treatment. Amongst other components not described, BCC treatment can incorporate: a laser, with display, controller and 1064 nm laser module, 1064 nm laser scanner (galvos drive beam position), thermal camera, RGB camera, LED illumination, stainless steel standoff for laser scanner, secondary screen for treatment viewing, imaging targets, laser microprocessors, and a computer (to handles camera inputs, scripts for image processing).

In this example, various control systems and modules can be implemented for treating BCC and further for the calibration and control of the above listed components. For instance, the system may include thermal camera calibration modules (to adjust camera pixel or subregion intensity, account for environmental variables, and enable object temperature imaging), and image registration modules (enabling the overlay of a thermal image on a visible image for operator visibility, i.e., a homography technique) thereby allowing a user to see the treatment in real time and conduct the treatment, for example, on a secondary screen. The system can further include coordinate system mapping modules (for converting “subregion space” or “pixel space” from camera images into “real space” distance and dimensioning on the tissue). Camera and image processing engines and modules can provide feedback in pixel or subregion location. Galvos that steer the beam can use applied voltages, so the beam can be moved to a desired location. Beam localization and position compensation modules can be incorporated to account for variation in beam trajectory due to instrument configuration. This will utilize the aiming beam to help ensure the alignment between the coordinate system and the laser beam location for each treatment. The system can further include a contour finding module (computer parses images to identify and trace a marker region drawn or painted on the tissue by the doctor or clinician, indicating desired treatment zone). This device does not necessarily diagnose but can help guide the operator toward complete treatment after the physician decides what should be treated. The system can further include a path planning module (that divides the contour into a number of spots and decide spot order and travel path). In the initial treatment this path can determine the first treatment pass to begin a control loop.

This utilizes the above contour and coordinate system mapping. The system can further include patient motion tracking (for instance as a safety feature) which may be implemented in an “interlock” style which can use image monitoring of visible wavelength camera to determine if contour (patient) moves. The system can further include a core control loop which contains thermal video feed (sensor), image processing (heating over time), accumulation matrix for Arrhenius rate at each location (spatial tracking), current temperature matrix of physical treatment locations, decision tree (where to go next, such as a minimum temp location/sROI, and radiation dose intensity to place each sROI within the therapeutic window). The system can further include a driving rate or rate function, as described further herein. Instead of using exact temperature or even expression of temperature over time, this equation converts sensed tissue response into a likelihood or other partial probability of treatment outcome (e.g., cell death), such that the spatially resolved accumulator matrix tracks this likelihood for each location. This can be used to treat to completion at all locations and drive the control loop that decides where to treat next between available locations mid-treatment. In this example embodiment, the Arrhenius equation is implemented as a rate function to convert sensed temperature in each frame for each sROI into an accumulation of likelihood of cell death and then use this to 1) track each location and ensure all are treated to completion 2) ensure temperature remains within a therapeutic window to prevent adverse events, and 3) provide custom tailored treatment in real time to account for differential tissue response within and around the lesion.

In another example, production level tasks may be incorporated into the system or method and can include: align focus and calibrate both thermal and RGB cameras and visible light sources; perform and confirm homography image registration between thermal and RGB camera, and store the homography transfer matrix to memory (automatic with error minimization algorithm to optimize settings common to registration); implement coordinate system mapping (converting “subregion space” into “real space”) to map the coordinate system of each camera to real space using information from a target and the standoff (it can be expected subregion space to real space to be non-uniform in x and y due to angular offset from target); perform beam localization and position compensation to account for variations in beam trajectory due to instrument configuration and can further include tasks that locate the aiming beam centroid at a defined location or list of defined locations (often treatment contour or target if at production), compute the beam position “offsets” and loop until accurate, test the offset protocol against a defined target in which the system steers the aiming beam atop a series of targets.

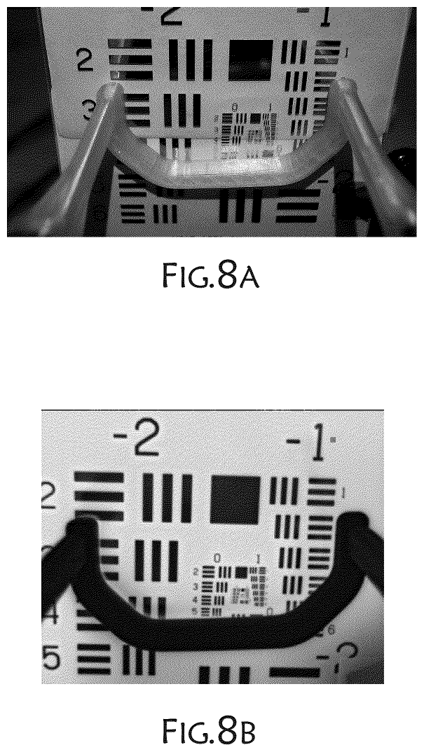

Certain steps of the foregoing example implementation are illustrated in more detail by FIGS. 8-16. Specifically, a thermal camera, an RGB camera, and sources of visible light were aligned and calibrated as described above. FIG. 8A illustrates the two-dimensional output (detected image) of an RGB camera (imaging device) oriented in a treatment device (not shown). The RGB camera is positioned in a treatment device in a manner similar to the positioning of the imaging device 406 in the treatment device 402 of FIG. 4. In FIG. 8A, an applicator component or standoff component similar to applicator component 416 of FIG. 4 is visible in the detected image. An Air Force 1951 style resolution target is visible adjacent to (or “behind”) the applicator component or standoff. FIG. 8B illustrates the two-dimensional output (detected temperature) of a thermal camera (tissue response detector) oriented in a treatment device (not shown) in a manner similar to tissue response detector 404 in treatment device 402 of FIG. 4, directed to the same resolution target as in FIG. 8A. The tissue response detector whose output is shown in FIG. 8B is present in the same overall treatment device as the imaging device whose output is shown in FIG. 8A. In FIG. 8B, the same applicator component or standoff component as in FIG. 8A is visible.

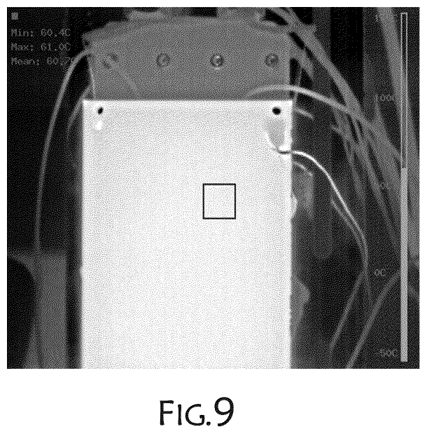

FIG. 9 illustrates a two-dimensional image of a calibration rig or setup captured by the same thermal camera described in FIG. 8. The calibration rig or setup of FIG. 9 is a black body radiator whose temperature can be varied as desired to create a calibration surface for the RGB camera and thermal camera of FIG. 8. The square near the middle of FIG. 9 marks a two-dimensional spatial region whose temperature is detected by the thermal camera over time (which can be referred to as a calibration region). The minimum, maximum, and mean temperature of this calibration region at a given point in time are shown in FIG. 9. The precise structure of the calibration rig is not particularly important—the wires and other features shown in FIG. 9 are not significant for the calibration process, provided the temperature of the calibration region can be tracked over time, as understood by one of ordinary skill in the art.

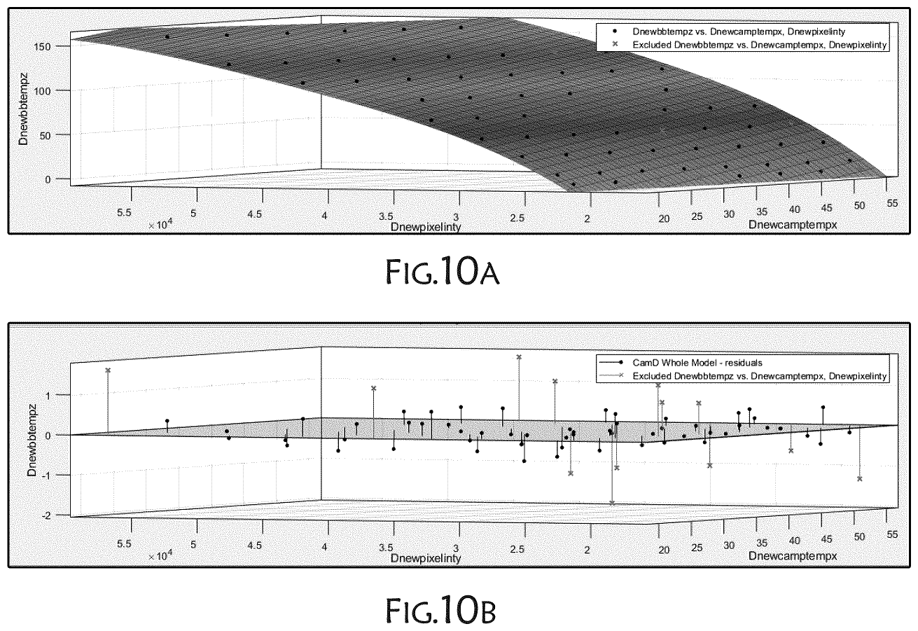

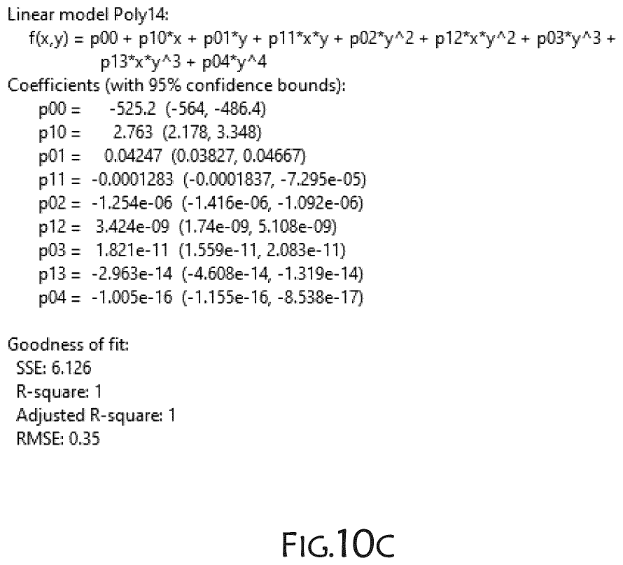

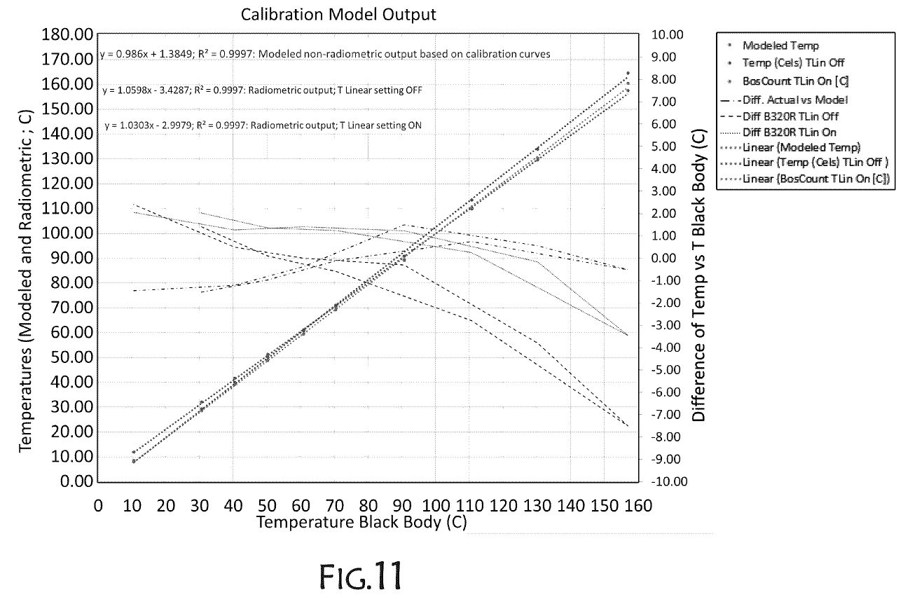

FIGS. 10A-C illustrate further steps of the alignment and calibration process. The temperature of the calibration region of FIG. 9 is changed over time, with stabilization in between desired temperature points (where stabilization may be achieved, for instance, by waiting for 5 minutes). In FIG. 10, the desired temperature points span physiologically relevant temperatures. FIG. 10A illustrates a three-dimensional plot of ordered triplets corresponding to the detected temperature of a pixel within the calibration region using the thermal camera, the temperature of the thermal camera itself, and the temperature of black body radiator itself, measured differently than with the thermal camera. The plot of FIG. 10A can be referred to as a calibration surface. FIG. 10B illustrates a plot of error or outlier analysis, and the ordered triplets indicated with an “x” in both FIG. 10A and FIG. 10B were eliminated as outliers. FIG. 10C illustrates a modeled polynomial based on the calibration surface of FIG. 10A. FIG. 11 illustrates a plot of the output of the calibration process.

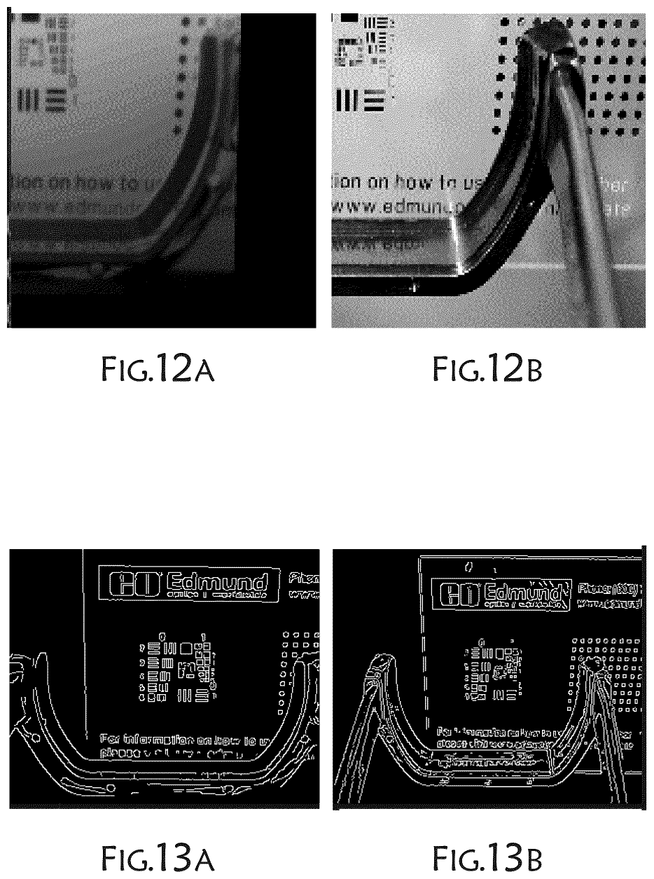

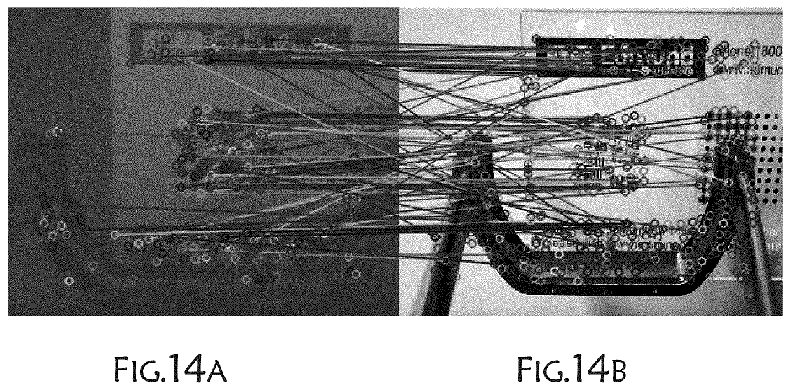

FIGS. 12-14 further illustrate aspect of performance of homography image registration between the thermal camera and RGB camera. More specifically, FIG. 12A and FIG. 12B illustrate the output images of the thermal camera (FIG. 12A) and RGB camera (FIG. 12B), with reference to the same calibration target (Air Force 1951) as described above. After the output images are taken, they are resized, normalized to intensity, filtered, and/or thresholded. In addition, as illustrated in FIG. 13A and FIG. 13B, edge detection is performed on both images. FIG. 13A illustrates the thermal camera output image, and FIG. 13B illustrates the RGB camera output image. FIG. 14 illustrates feature matching based on random sample consensus of edges (rejecting bad matches). In FIG. 14, the features from the thermal camera output image (FIG. 14A) are matched to the features from the RGB camera output image (FIG. 14B). In this example, homography (or rigid registration, or affine transformations, as may be required or preferred depending on system optics) is used to register and overlay images from the thermal camera and RGB camera outputs. Further, the homography coordinates are stored to memory of the system (as described above, for instance).

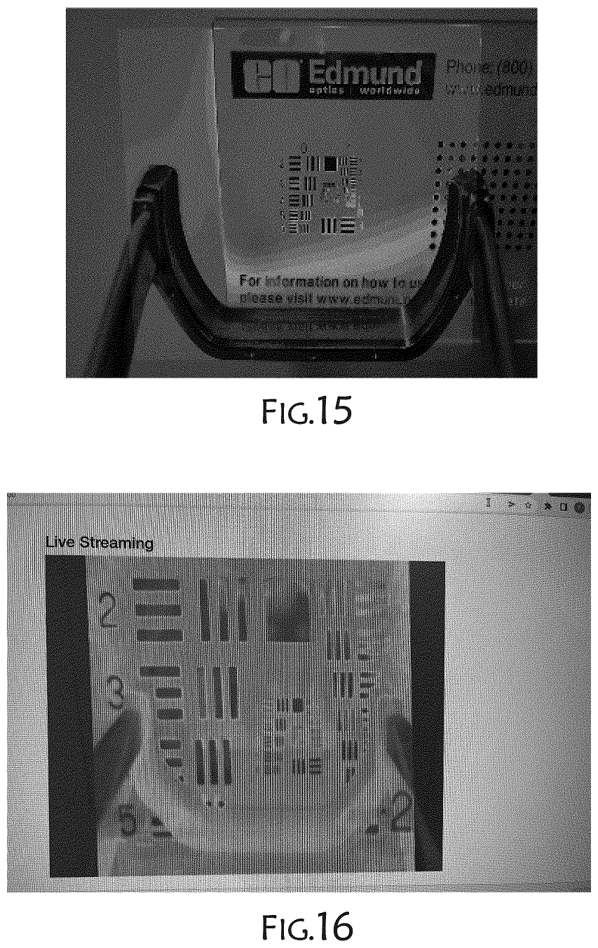

Next, in this present example embodiment, defined thermal gradients and/or isotherms are then used to show overlay of thermal and RGB camera images, executed in real time during treatment and calibration using stored coordinates. See FIG. 15 for an overlay having good registration. The combined images can also be streamed or otherwise provided continuously and/or simultaneously to provide real time visualization (e.g., on a secondary screen or monitor) for physician guidance and safety. FIG. 16 illustrates a live streamed image of a calibrated thermal camera imaging a heated Air Force 1951 target in thermal imaging mode, as displayed over WiFi LAN on a secondary screen.

Another example treatment method comprises: scanner calibration is checked and confirmed, physician encircles the lesion plus a clinical margin with a tissue pen, scanner standoff is placed on the tissue centered over the lesion, camera scans the lesion margin and IDs the lesion border. The laser then traces the border with the aiming beam. The physician confirms the correct area is highlighted. Then, the laser calculates a path for the beam to scan over the whole enclosed area and treats the area with a “first pass” to elevate the temperature and begin the control loop. The control loop then processes the steps of: the location of the lesion is tracked to ensure the patient has not moved relative to the treatment beam. Correct if needed or fail the treatment if needed for safety. Aim beam location is checked relative to desired location and corrected if needed. The tissue response (temperature) for each sROI is calculated. Temperature map is updated. The incremental likelihood of cell death in that area is calculated via Arrhenius rate equation. The matrix of cell death likelihood is updated at each location. The next location to treat is chosen based on temperature and likelihood of cell death (complete, i.e., likely dead areas are excluded), lowest remaining temperature location is selected, difference between current temperature and target temperature is calculated, pulse fluence is calculated to bring the “next target” location to the desired temperature. The laser moves to the next chosen location and doses at the prescribed fluence. The cameras check the tissue response and the loop iterates. An ending sequence can also be implemented which can include the steps of: all locations register as above the threshold of high likelihood of cell death, and the treatment data is recorded for review.

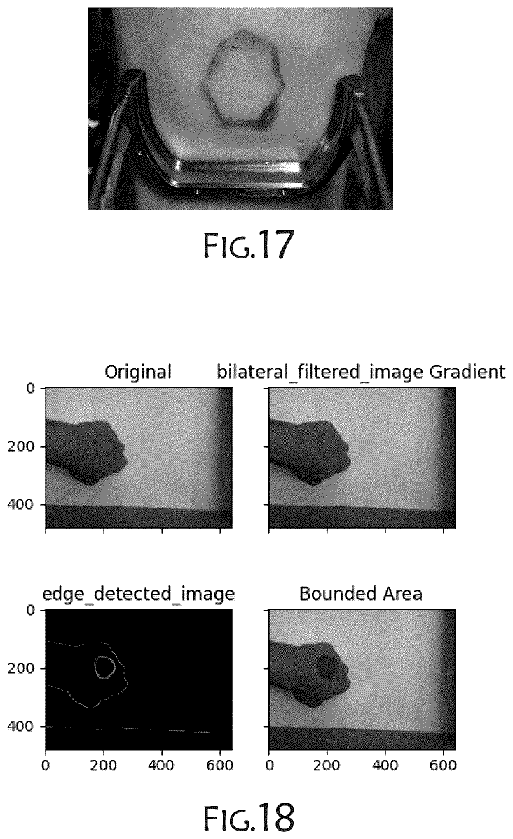

Certain steps of the foregoing example are further illustrated and described with reference to FIGS. 17-25. A system similar to that described hereinabove (e.g., in connection with FIGS. 4, 5, and 8-16) can be used in the context of FIGS. 17-25.

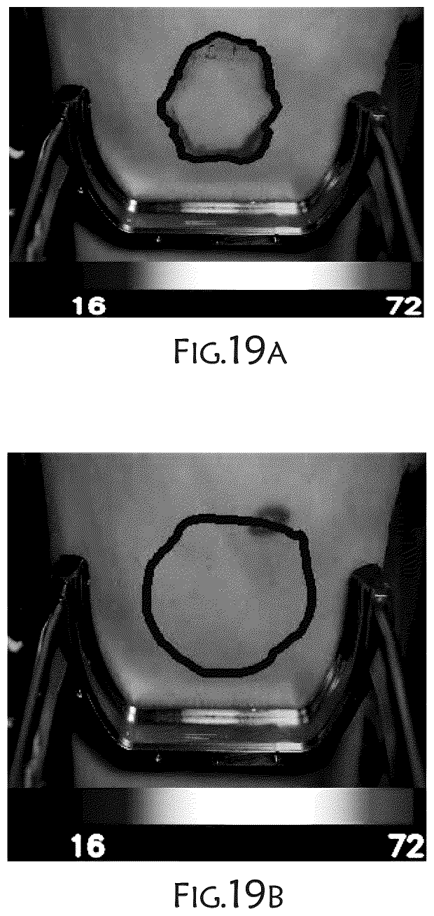

With reference to FIG. 17, this figure shows an RGB image (e.g., captured by an RGB camera imaging device as described above) including an example of how a physician can encircle, define, or otherwise identify a pathological region of skin and a clinically relevant margin with a skin-marking pen or marker (see the generally hexagonal perimeter marked on the skin in FIG. 17). FIG. 18 illustrates an encircled region on a hand of a patient, in which image registration (e.g., as described above in connection with FIGS. 8-16) identifies the skin and marker and uses filtering and edge detection. FIG. 19A illustrates overlaid thermal and RGB images and shows a computer generated trace of outer bounds of treatment area (e.g., ROI) on the skin. In an alternative embodiment (FIG. 19B), the physician may choose to draw on a touchscreen interface of the images, instead of using a physical or traditional hand-held marker on the skin.

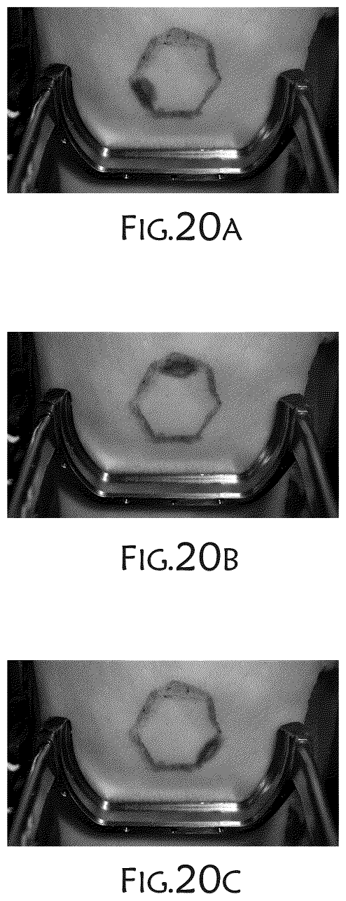

Returning again to the example implementation of FIG. 17, once the border is drawn and the system recognizes the marking, the laser traces the defined border and the physician confirms the area is correctly identified, also confirming system alignment. FIGS. 20A-C illustrate the laser tracing the edge of the region of FIG. 17, sequentially in each frame. In FIG. 20A, the laser spot is seen in the lower left quadrant of the border defined by the marked region. In FIG. 20B, the laser is seen in the top center of the border defined by the marked region. In the FIG. 20C, the laser is seen in the lower right quadrant of the border defined by the marked region.

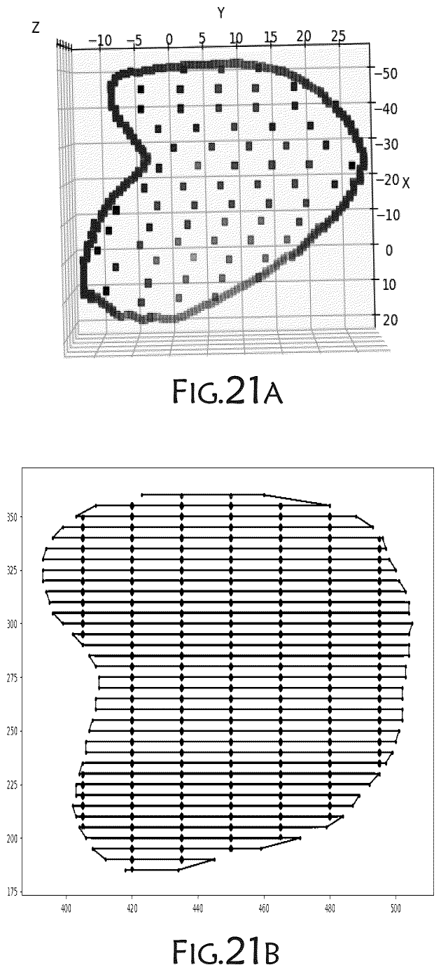

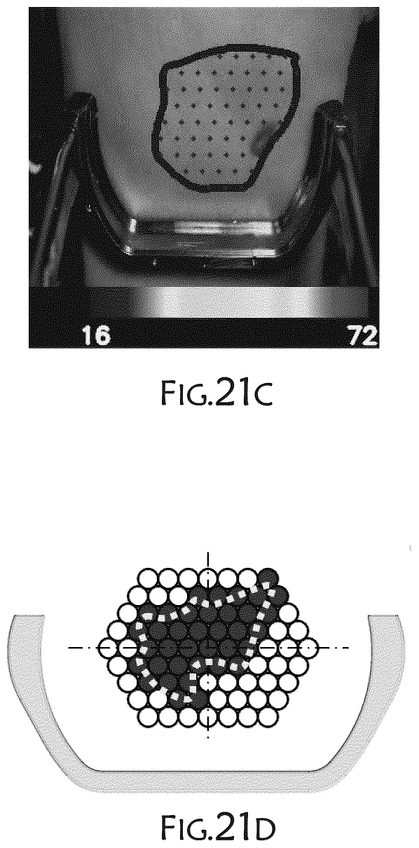

In this specific non-limiting example embodiment, after the ROI is confirmed by the physician, the computer system then plans a path for the initial passes of treatment, both in the directions that the laser galvos will scan through the desired space, as well as when in the pattern the laser will fire. This process is illustrated by FIGS. 21A-D. FIG. 21A illustrates a computer illustration of a region border as detected. Spots inside the border correspond to planned laser pulse deposition (or ‘firing’) locations within the ROI. FIG. 21B illustrates waypoints for galvo scans through the pattern of FIG. 21A, adjusted for known laser beam diameter or spot size. FIG. 21C illustrates a different region (ROI), as drawn by a physician on a touchscreen, showing the border and planned treatment locations overlaid in a live image feed. As illustrated in FIG. 21D, an alternative strategy for planning may include known beam locations that encompass the margin line. FIG. 21D shows the u-shaped standoff (or applicator component) and potential beam locations, as well as a dotted line for the drawn margin, and the filled beam locations that are utilized to deliver the treatment energy.

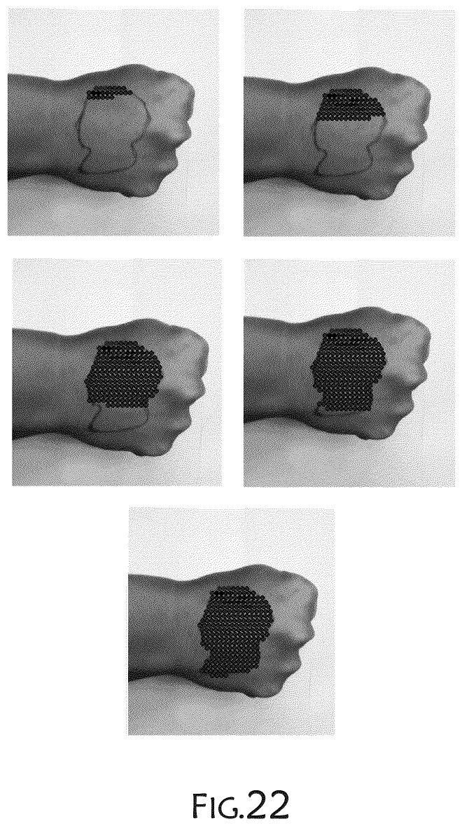

FIG. 22 illustrates an alternative embodiment of a planned path of treatment. As illustrated in FIG. 22 (from top to bottom), a planned raster pattern ‘fills’ the ROI on the hand of a patient with sequential irradiation by a laser, as part of the initial irradiation of multiple sROIs.

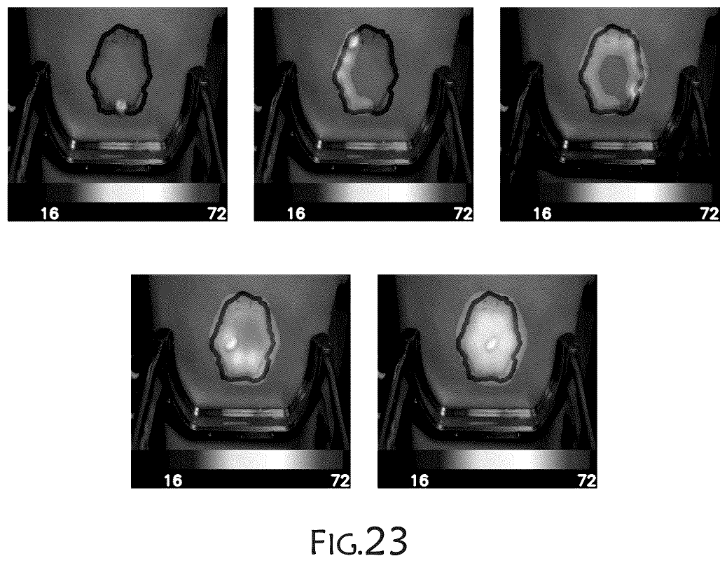

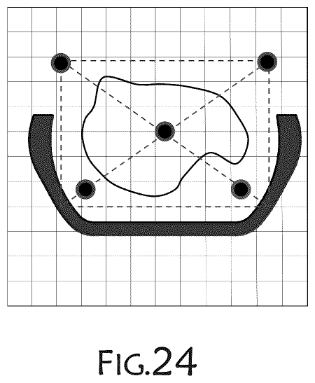

FIG. 23 further illustrates steps of a treatment method. With reference to FIG. 23, in sequence of left to right, top to bottom, the first pass of treatment determines the tissue response to a known dose for each individual unit area (e.g., sROI) within the defined and confirmed treatment region (e.g., the ROI). In the embodiment of FIG. 23, the deposited or applied energy causes a temperature increase that exists beyond a single camera frame. That is, the temperature increase caused by an initial dose at a given pixel or sROI extends in time from frame to frame in FIG. 23. During this and all portions of treatment in the embodiment of FIG. 23, the RGB image and the thermal image are processed to determine the cumulative time-resolved doses, as described herein. Initial passes both bring the tissue into the target therapeutic window (e.g., based on a rate equation) and help to determine any variability in tissue response due to many factors, such as minor fluctuations in energy delivery, differences in heat generated per unit energy delivered, and variations in the thickness or severity of pathology at each location (e.g., sROI). Each of the foregoing sources of variability could potentially be anticipated but not likely accurately known in advance of providing treatment. Based on the methods described herein, subsequent exposures, ‘passes’, or irradiation of one or more sROIs can provide the desired therapeutic effect or degree of treatment completion.

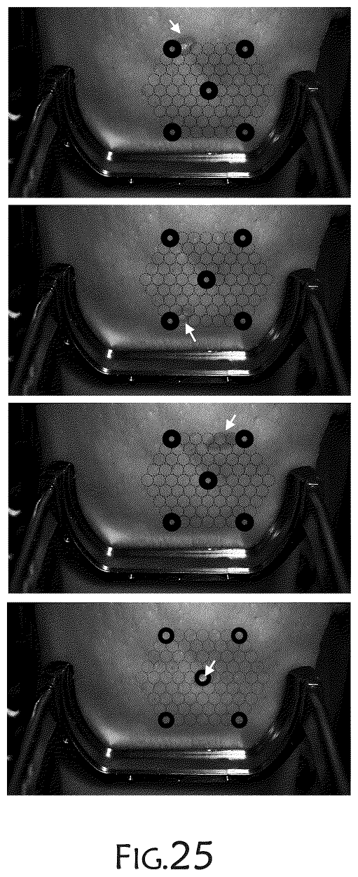

FIGS. 24 and 25 further illustrate steps of the example treatment method. FIG. 24 illustrates schematically an ROI (the amorphous perimeter in FIG. 24), with reference a u-shaped standoff or applicator component described herein. In accordance with methods described herein, a scanner may routinely make aiming beam alignment illuminations (dots in FIG. 24) which are recognized by an RGB camera as described herein. The location of the aiming beam relative to the standoff and the defined region of interest are checked to be within known bounds and corrected to ensure proper treatment, minimizing the impact of slight movements by the patient or the physician guiding the laser placement onto the skin. FIG. 25 illustrates RGB outputs of a similar ROI as depicted schematically in FIG. 24.

More specifically, FIG. 25 illustrates RGB images of the skin and the aiming beam as well as a projected overlay of available treatment space (dotted circles in FIG. 25). Each RGB output of FIG. 25 illustrates the laser aimed at one of the target circles. However, in three of the RGB outputs (the top three in FIG. 25), the laser is not perfectly targeting the desired target circle (which each may be an sROI, for instance). The white arrows in FIG. 25 show the center of the actual laser spot in each instance. Therefore, as described herein, the method of the present example compensates for this misalignment (based on the tissue response detected) and compensates for the ‘miss’ in subsequent treatment steps.

Some additional non-limiting example Embodiments are described below.

Embodiment 1. A method of treating biological tissue of a patient in need thereof, the method comprising:
irradiating a region of interest (ROI) of the patient with one or more initial doses of electromagnetic radiation, wherein the ROI comprises a matrix or array of sub regions of interest (sROIs) of biological tissue of the patient;
determining a tissue response of one or more of the sROIs;
generating a tissue response matrix from the tissue response of the one or more sROIs;
translating the tissue response matrix into a sub-clinical indicator matrix or treatment completion matrix for the one or more sROIs, the sub-clinical indicator matrix corresponding to a degree of treatment completion for the one or more sROIs; and
irradiating one or more sROIs or no sROIs with one or more additional doses of electromagnetic radiation based at least in part on the sub-clinical indicator matrix.

Embodiment 2. The method of Embodiment 1, wherein the sub-clinical indicator matrix comprises an aggregation of degrees of treatment completion for the one or more sROIs over time.

Embodiment 3. The method of Embodiment 1 or Embodiment 2, wherein two or more of the irradiating, determining, generating, and translating steps occur simultaneously, continuously, and/or in real time.

Embodiment 4. The method of any of the preceding Embodiments, wherein the one or more additional doses of electromagnetic radiation have the same or different properties as the one or more initial doses.

Embodiment 5. The method of any of the preceding Embodiments, wherein the sub-clinical indicator matrix is generated based on one or more rate functions.

Embodiment 6. The method of Embodiment 5, wherein the one or more rate functions comprises at least one of an Arrhenius function, a bioheat function, a light transport function, and a derivative of a light transport function.

Embodiment 7. The method of any of the preceding Embodiments, wherein the tissue response corresponds to a sensed temperature, a detected color or color change, or another detected spectral change.

Embodiment 8. The method of any of the preceding Embodiments, wherein each sROI corresponds to a unit-sized real physical location of the biological tissue with a known size and location relative to one or more other sROIs of the ROI.

Embodiment 9. The method of any of the preceding Embodiments, wherein the ROI corresponds to a defined treatment area.

Embodiment 10. The method of any of the preceding Embodiments, wherein the electromagnetic radiation is a laser beam.

Embodiment 11. The method of Embodiment 10, wherein the laser beam has an average wavelength in the ultraviolet (UV), visible, or infrared (IR) region of the electromagnetic spectrum, such as an average wavelength λ between 190 nm and 10.6 μm or between 190 nm and 3 μm.

Embodiment 12. The method of Embodiment 10, wherein the laser beam has an average wavelength λ between 700 nm and 1500 nm or between 900 and 1300 nm.

Embodiment 13. The method of Embodiment 10, wherein the laser beam comprises an Nd: YAG laser beam.

Embodiment 14. The method of any of the preceding Embodiments, wherein the degree of treatment completion comprises achieving a benchmark temperature over time for one or more sROIs.

Embodiment 15. The method of any of the preceding Embodiments, wherein the degree of treatment completion comprises achieving cell death of the biological tissue of one or more sROIs.

Embodiment 16. The method of any of the preceding Embodiments, the method further comprising imaging the ROI with an imaging device.

Embodiment 17. The method of Embodiment 16, wherein the ROI is imaged in real time.

Embodiment 18. The method of any of the preceding Embodiments, the method further comprising irradiating the matrix of sROIs a sufficient number of times to complete the treatment.

Embodiment 19. A system for treating biological tissue of a patient, the system comprising:
a source of electromagnetic radiation to irradiate a region of interest (ROI) of the patient with one or more initial doses of electromagnetic radiation, wherein the ROI comprises a matrix or array of sub regions of interest (sROIs) of biological tissue of the patient;
an imaging device;
a tissue response detector to determine a tissue response of one or more of the sROIs based on the irradiation of the ROI; and
a control device to generate a tissue response matrix based on information received from the tissue response detector, and to translate the tissue response matrix into a sub-clinical indicator matrix or treatment completion matrix for the one or more sROIs,
wherein the sub-clinical indicator matrix corresponds to a degree of treatment completion for the one or more sROIs; and
wherein the control device further signals (directly or indirectly) the source of electromagnetic radiation to irradiate one or more sROIs or no sROIs with one or more additional doses of electromagnetic radiation based at least in part on the sub-clinical indicator matrix.

Embodiment 20. The system of Embodiment 19, wherein the sub-clinical indicator matrix comprises an aggregation of degrees of treatment completion for the one or more sROIs over time.

Embodiment 21. The system of Embodiment 19 or Embodiment 20, wherein the one or more additional doses of electromagnetic radiation have the same or different properties as the one or more initial doses.

Embodiment 22. The system of any one of Embodiments 19-21, wherein the sub-clinical indicator matrix is generated based on one or more rate functions.

Embodiment 23. The system of Embodiment 22, wherein the one or more rate functions comprises at least one of an Arrhenius function, a bioheat function, and a light transport function.

Embodiment 24. The system of any one of Embodiments 19-23, wherein the tissue response detector is configured to detect a temperature of one or more sROIs.

Embodiment 25. The system of any of Embodiments 19-24, wherein the source of electromagnetic radiation is a Nd: YAG laser.

Embodiment 26. The system of any of Embodiments 19-25, wherein the source of electromagnetic radiation has an average wavelength in the ultraviolet (UV), visible, or infrared (IR) region of the electromagnetic spectrum, such as an average wavelength λ between 190 nm and 10.6 μm, between 190 nm and 3 μm, between 700 nm and 1500 nm, or between 900 and 1300 nm Embodiment 27. The system of any of Embodiments 19-26, wherein the imaging device is configured to image one or more of the sROIs.

Embodiment 28. The system of Embodiment 27, wherein the imaging is done in real time.

Embodiment 29. A computer storage medium storing computer-useable instructions that, when used by one or more computing devices, cause the one or more computing devices to treat biological tissue of a patient, the operations comprising:
irradiating a region of interest (ROI) of the patient with one or more initial doses of electromagnetic radiation, wherein the ROI comprises a matrix or array of sub regions of interest (sROIs) of biological tissue of the patient;
determining a tissue response of one or more of the sROIs;
generating a tissue response matrix from the tissue response of the one or more sROIs;
translating the tissue response matrix into a sub-clinical indicator matrix or treatment completion matrix for the one or more sROIs, the sub-clinical indicator matrix corresponding to a degree of treatment completion for the one or more sROIs; and
irradiating one or more sROIs or no sROIs with one or more additional doses of electromagnetic radiation based at least in part on the sub-clinical indicator matrix.

Embodiment 30. The computer storage medium of Embodiment 29, wherein the operations further comprise mapping a plurality of sROIs of the tissue.

Embodiment 31. The computer storage medium of Embodiment 29 or Embodiment 30, wherein the sub-clinical indicator matrix comprises an aggregation of degrees of treatment completion for the one or more sROIs over time.

Embodiment 32. The computer storage medium of any one of Embodiments 29-31, wherein two or more of the irradiating, determining, generating, and translating steps occur simultaneously, continuously, and/or in real time.

Embodiment 33. The computer storage medium of any one of Embodiments 29-32, wherein the one or more additional doses of electromagnetic radiation have the same or different properties as the one or more initial doses.

Embodiment 34. The computer storage medium of any one of Embodiments 29-33, wherein the sub-clinical indicator matrix is generated based on one or more rate functions.

Embodiment 35. The computer storage medium of Embodiment 34, wherein the one or more rate functions comprises at least one of an Arrhenius function, a bioheat function, and a light transport function.

Embodiment 36. The computer storage medium of any of Embdiments 29-35, wherein the tissue response corresponds to a sensed temperature.

Many different arrangements of the various components and/or steps depicted and described, as well as those not shown, are possible without departing from the scope of the claims below. Embodiments of the present technology have been described with the intent to be illustrative rather than restrictive. Alternative embodiments will become apparent from reference to this disclosure. Alternative means of implementing the aforementioned can be completed without departing from the scope of the claims below. Certain features and subcombinations are of utility and can be employed without reference to other features and subcombinations and are contemplated within the scope of the claims.

1. A method of treating biological tissue of a patient in need thereof, the method comprising:
irradiating a region of interest (ROI) of the patient with one or more initial doses of electromagnetic radiation, wherein the ROI comprises a matrix of sub regions of interest (sROIs) of biological tissue of the patient;
determining a tissue response of one or more of the sROIs;
generating a tissue response matrix from the tissue response of the one or more sROIs;
translating the tissue response matrix into a sub-clinical indicator matrix for the one or more sROIs, the sub-clinical indicator matrix corresponding to a degree of treatment completion for the one or more sROIs; and
irradiating one or more sROIs or no sROIs with one or more additional doses of electromagnetic radiation based at least in part on the sub-clinical indicator matrix.

2. The method of claim 1, wherein the sub-clinical indicator matrix comprises an aggregation of degrees of treatment completion for the one or more sROIs over time.

3. The method of claim 1, wherein two or more of the irradiating, determining, generating, and translating steps occur simultaneously, continuously, and/or in real time.

4. The method of claim 1, wherein the one or more additional doses of electromagnetic radiation have the same or different properties as the one or more initial doses.

5. The method of claim 1, wherein the sub-clinical indicator matrix is generated based on one or more rate functions.

6. The method of claim 5, wherein the one or more rate functions comprises at least one of an Arrhenius function, a bioheat function, and a light transport function.

7. The method of claim 1, wherein the tissue response corresponds to a sensed temperature, a detected color or color change, or another detected spectral change.

8. The method of claim 1, wherein each sROI corresponds to a unit-sized real physical location of the biological tissue with a known size and location relative to one or more other sROIs of the ROI.

9. The method of claim 1, wherein the ROI corresponds to a defined treatment area.

10. The method of claim 1, wherein the electromagnetic radiation is a laser beam.

11. The method of claim 10, wherein the laser beam has an average wavelength λ between 190 nm and 10.6 μm.

12. The method of claim 10, wherein the laser beam has an average wavelength λ between 700 nm and 1500 nm.

13. The method of claim 10, wherein the laser beam comprises an Nd: YAG laser beam.

14. The method of claim 1, wherein the degree of treatment completion comprises achieving a benchmark temperature over time for one or more sROIs.

15. The method of claim 1, wherein the degree of treatment completion comprises achieving cell death of the biological tissue of one or more sROIs.

16. The method of claim 1, the method further comprising imaging the ROI with an imaging device.

17. The method of claim 16, wherein the ROI is imaged in real time.

18. The method of claim 1, the method further comprising irradiating the matrix of sROIs a sufficient number of times to complete the treatment.

19. A system for treating biological tissue of a patient, the system comprising:
a source of electromagnetic radiation to irradiate a region of interest (ROI) of the patient with one or more initial doses of electromagnetic radiation, wherein the ROI comprises a matrix of sub regions of interest (sROIs) of biological tissue of the patient;
an imaging device;
a tissue response detector to determine a tissue response of one or more of the sROIs based on the irradiation of the ROI; and
a control device to generate a tissue response matrix based on information received from the tissue response detector, and to translate the tissue response matrix into a sub-clinical indicator matrix for the one or more sROIs,
wherein the sub-clinical indicator matrix corresponds to a degree of treatment completion for the one or more sROIs; and
wherein the control device further signals the source of electromagnetic radiation to irradiate one or more sROIs or no sROIs with one or more additional doses of electromagnetic radiation based at least in part on the sub-clinical indicator matrix.

20-28. (canceled)

29. A computer storage medium storing computer-useable instructions that, when used by one or more computing devices, cause the one or more computing devices to treat biological tissue of a patient, the operations comprising:
irradiating a region of interest (ROI) of the patient with one or more initial doses of electromagnetic radiation, wherein the ROI comprises a matrix of sub regions of interest (sROIs) of biological tissue of the patient;
determining a tissue response of one or more of the sROIs;
generating a tissue response matrix from the tissue response of the one or more sROIs;
translating the tissue response matrix into a sub-clinical indicator matrix for the one or more sROIs, the sub-clinical indicator matrix corresponding to a degree of treatment completion for the one or more sROIs; and
irradiating one or more sROIs or no sROIs with one or more additional doses of electromagnetic radiation based at least in part on the sub-clinical indicator matrix.

30-36. (canceled)
